(19) United States
(12) Patent Application Publication
(10) Pub. No.: US 2021/0244601 A1
(43) Pub. Date: Aug. 12, 2021

(54) WALKING ASSISTANCE SYSTEM, WALKING ASSISTANCE METHOD, AND WALKING ASSISTANCE PROGRAM

(71) Applicant: TOYOTA JIDOSHA KABUSHIKI KAISHA, Toyota-shi (JP)

(72) Inventor: Tomoe MAEKITA, Toyota-shi (JP)

(73) Assignee: TOYOTA JIDOSHA KABUSHIKI KAISHA, Toyota-shi (JP)

(21) Appl. No.: 17/121,910

(22) Filed: Dec. 15, 2020

(30) Foreign Application Priority Data
Feb. 12, 2020 (JP) 2020-021461

(51) Int. Cl. A61H 3/00; A61B 5/11

(52) U.S. Cl. CPC A61H 3/008; A61B 5/112; A61H 2003/007; A61H 2205/102; A61H 2201/0165; A61H 2201/164; A61H 2203/0406; A61H 2201/5058

(57) ABSTRACT

A walking assistance system according to an embodiment includes: a damper that applies a resisting force in a bending direction of a knee joint of the leg; extending assistance means for applying an assisting force in an extending direction of the knee joint; a sensor that detects a switching timing in a gait cycle of the user; and a control unit that switches modes of the damper in accordance with the switching timing so that a first bending mode and a second bending mode in which a resisting force larger than that in the first bending mode is applied are alternately repeated and switches modes of the extending assistance means in accordance with the switching timing so that a first extending mode and a second extending mode in which an assisting force larger than that in the first extending mode is applied are alternately repeated.

### CROSS REFERENCE TO RELATED APPLICATIONS

This application is based upon and claims the benefit of priority from Japanese patent application No. 2020-021461, filed on Feb. 12, 2020, the disclosure of which is incorporated herein in its entirety by reference.

### BACKGROUND

The present disclosure relates to a walking assistance system, a walking assistance method, and a walking assistance program.

Japanese Unexamined Patent Application Publication No. 2019-005250 discloses a walking assistance apparatus that assists a walking motion. The walking assistance apparatus disclosed in Japanese Unexamined Patent Application Publication No. 2019-005250 assists a user in walking by controlling a trajectory of the knee joint of the user in a two-cycle motion including a stance phase and a swing phase.

### SUMMARY

When a person who walks while bending his/her knees, a person whose knees are already bent in an initial stage of the stance phase or the like uses a walking assistance apparatus, a strong moment torque may be applied to his/her lower limbs from the initial stage of the swing phase. Such a case causes a problem that the capacity of a damper for generating a resisting force to a rotation of the knee joint in the direction in which the knee joint is bent is exceeded, and thus the knee is easily bent.

The present disclosure has been made to solve the aforementioned problem, and it provides a walking assistance system, a walking assistance method, and a walking assistance program that are capable of preventing an excessive knee bending.

A first exemplary aspect is a walking assistance system attached to a leg of a user, including: a damper configured to apply a resisting force in a bending direction of a knee joint of the leg; extending assistance means for applying an assisting force in an extending direction of the knee joint; a sensor configured to detect a switching timing in a gait cycle of the user; and a control unit configured to switch modes of the damper in accordance with the switching timing so that a first bending mode and a second bending mode in which a resisting force larger than that in the first bending mode is applied are alternately repeated and switch modes of the extending assistance means in accordance with the switching timing so that a first extending mode and a second extending mode in which an assisting force larger than that in the first extending mode is applied are alternately repeated, in which in a swing phase during which a sole of a foot of the leg comes off a ground, the control unit switches the mode of the extending assistance means from the first extending mode to the second extending mode at least in a later stage of an extending phase during which the knee joint comes close to an extended state.

In the aforementioned walking assistance system, the control unit may be configured to be able to adjust a start timing of the second extending mode in accordance with a degree of a bending of the knee joint of the user.

In the aforementioned walking assistance system, the control unit may be configured to be able to adjust the start timing of the second extending mode by setting it in advance.

In the aforementioned walking assistance system, the control unit may be configured to be able to adjust the start timing of the second extending mode based on a walking state detected by the sensor.

In the aforementioned walking assistance system, the sensor may include any one of: a gyro sensor configured to detect an angle of a lower leg with respect to the ground; an angle sensor configured to detect an angle formed by an upper leg and the lower leg around the knee joint; an angle sensor configured to detect a bending angle at which the lower leg is bent from a state in which the knee joint is extended; an angular velocity sensor configured to detect an angular velocity of each of the angles or an angular velocity of the bending angle; a distance measuring sensor configured to detect a distance between a predetermined position of the leg and the ground; a ground contact timing sensor configured to detect a timing at which the sole of the foot comes into contact with the ground; and an imaging sensor configured to shoot the leg.

In the aforementioned walking assistance system, the second bending mode may include a period in which a magnitude of the resisting force is gradually increased from that of the resisting force in the first bending mode.

In the aforementioned walking assistance system, the second extending mode may include a period in which a magnitude of the assisting force is gradually increased from that of the assisting force in the first extending mode.

In the aforementioned walking assistance system, the extending assistance means may include an actuator or an elastic body.

In the aforementioned walking assistance system, in a stance phase during which the sole of the foot is in contact with the ground, the first extending mode or the second extending mode may be set in the extending assistance means.

In the aforementioned walking assistance system, in the extending phase, the first bending mode or the second bending mode may be set in the damper.

Another exemplary aspect is a walking assistance method using a walking assistance apparatus attached to a leg of a user, the walking assistance apparatus including: a damper configured to apply a resisting force in a bending direction of a knee joint of the leg; extending assistance means for applying an assisting force in an extending direction of the knee joint; and a sensor configured to detect a switching timing in a gait cycle of the user, the walking assistance method including switching modes of the damper in accordance with the switching timing so that a first bending mode and a second bending mode in which a resisting force larger than that in the first bending mode is applied are alternately repeated and switching modes of the extending assistance means in accordance with the switching timing so that a first extending mode and a second extending mode in which an assisting force larger than that in the first extending mode is applied are alternately repeated, in which in a swing phase during which a sole of a foot of the leg comes off a ground, the mode of the extending assistance means is switched from the first extending mode to the second extending mode at least in a later stage of an extending phase during which the knee joint comes close to an extended state.

In the walking assistance method, a start timing of the second extending mode may be adjustable in accordance with a degree of a bending of the knee joint of the user.

In the aforementioned walking assistance method, the start timing of the second extending mode may be adjustable by setting it in advance.

In the aforementioned walking assistance method, the start timing of the second extending mode may be adjustable based on a walking state detected by the sensor.

In the aforementioned walking assistance method, the sensor may include any one of: a gyro sensor configured to detect an angle of a lower leg with respect to the ground; an angle sensor configured to detect an angle formed by an upper leg and the lower leg around the knee joint; an angle sensor configured to detect a bending angle at which the lower leg is bent from a state in which the knee joint is extended; an angular velocity sensor configured to detect an angular velocity of each of the angles or an angular velocity of the bending angle; a distance measuring sensor configured to detect a distance between a predetermined position of the leg and the ground; a ground contact timing sensor configured to detect a timing at which the sole of the foot comes into contact with the ground; and an imaging sensor configured to shoot the leg.

In the aforementioned walking assistance method, the second bending mode may include a period in which a magnitude of the resisting force is gradually increased from that of the resisting force in the first bending mode.

In the aforementioned walking assistance method, the second extending mode may include a period in which a magnitude of the assisting force is gradually increased from that of the assisting force in the first extending mode.

In the aforementioned walking assistance method, the extending assistance means may include an actuator or an elastic body.

In the aforementioned walking assistance method, in a stance phase during which the sole of the foot is in contact with the ground, the first extending mode or the second extending mode may be set in the extending assistance means.

In the aforementioned walking assistance method, in the extending phase, the first bending mode or the second bending mode may be set in the damper.

Another exemplary aspect is a walking assistance program using a walking assistance apparatus attached to a leg of a user, the walking assistance apparatus including: a damper configured to apply a resisting force in a bending direction of a knee joint of the leg; extending assistance means for applying an assisting force in an extending direction of the knee joint; and a sensor configured to detect a switching timing in a gait cycle of the user, the walking assistance program causing a computer to switch modes of the damper in accordance with the switching timing so that a first bending mode and a second bending mode in which a resisting force larger than that in the first bending mode is applied are alternately repeated and switch modes of the extending assistance means in accordance with the switching timing so that a first extending mode and a second extending mode in which an assisting force larger than that in the first extending mode is applied are alternately repeated, in which in a swing phase during which a sole of a foot of the leg comes off a ground, the mode of the extending assistance means is switched from the first extending mode to the second extending mode at least in a later stage of an extending phase during which the knee joint comes close to an extended state.

In the walking assistance program, a start timing of the second extending mode may be adjustable in accordance with a degree of a bending of the knee joint of the user.

In the aforementioned walking assistance program, the start timing of the second extending mode may be adjustable by setting it in advance.

In the aforementioned walking assistance program, the start timing of the second extending mode may be adjustable based on a walking state detected by the sensor.

In the aforementioned walking assistance program, the sensor may include any one of: a gyro sensor configured to detect an angle of a lower leg with respect to the ground; an angle sensor configured to detect an angle formed by an upper leg and the lower leg around the knee joint; an angle sensor configured to detect a bending angle at which the lower leg is bent from a state in which the knee joint is extended; an angular velocity sensor configured to detect an angular velocity of each of the angles or an angular velocity of the bending angle; a distance measuring sensor configured to detect a distance between a predetermined position of the leg and the ground; a ground contact timing sensor configured to detect a timing at which the sole of the foot comes into contact with the ground; and an imaging sensor configured to shoot the leg.

In the aforementioned walking assistance program, the second bending mode may include a period in which a magnitude of the resisting force is gradually increased from that of the resisting force in the first bending mode.

In the aforementioned walking assistance program, the second extending mode may include a period in which a magnitude of the assisting force is gradually increased from that of the assisting force in the first extending mode.

In the aforementioned walking assistance program, the extending assistance means may include an actuator or an elastic body.

In the aforementioned walking assistance program, in a stance phase during which the sole of the foot is in contact with the ground, the first extending mode or the second extending mode may be set in the extending assistance means.

In the aforementioned walking assistance program, in the extending phase, the first bending mode or the second bending mode may be set in the damper.

According to the above exemplary aspects, it is possible to provide a walking assistance system, a walking assistance method, and a walking assistance program that are capable of preventing an excessive knee bending.

The above and other objects, features and advantages of the present disclosure will become more fully understood from the detailed description given hereinbelow and the accompanying drawings which are given by way of illustration only, and thus are not to be considered as limiting the present disclosure.

### BRIEF DESCRIPTION OF DRAWINGS

FIG. 1 is a front view illustrating a walking assistance apparatus according to a first embodiment;

FIG. 2 is a side view illustrating the walking assistance apparatus according to the first embodiment;

FIG. 3 is a block diagram showing a control system of the walking assistance apparatus according to the first embodiment;

FIG. 4 is a block diagram showing another control system of the walking assistance apparatus according to the first embodiment;

FIG. 5 is a diagram illustrating a walking training system according to the first embodiment;

FIG. 6 is a diagram illustrating a walking motion and a timing at which modes are switched in a gait cycle using the walking assistance apparatus according to the first embodiment; and

FIG. 7 is a diagram illustrating a walking motion and a timing at which the modes are switched in the gait cycle using a walking assistance apparatus according to a second embodiment.

### DESCRIPTION OF EMBODIMENTS

Hereinafter, although the present disclosure will be described with reference to embodiments of the present disclosure, the present disclosure according to claims is not limited to the following embodiments. Further, all the components described in the following embodiments are not necessarily essential as means for solving problems. For the clarification of the description, the following description and the drawings may be omitted or simplified as appropriate. Throughout the drawings, the same components are denoted by the same reference signs and repeated descriptions will be omitted as appropriate.

### First Embodiment

A walking assistance apparatus (a walking assistance system) according to a first embodiment is described. The walking assistance apparatus according to this embodiment is, for example, attached to a leg including a knee joint of a trainee who does walking training. The trainee, who is a user, does walking training while the walking assistance apparatus is attached to his/her leg. First, a configuration of the walking assistance apparatus according to this embodiment is described. After that, an operation of the walking assistance apparatus will be described.

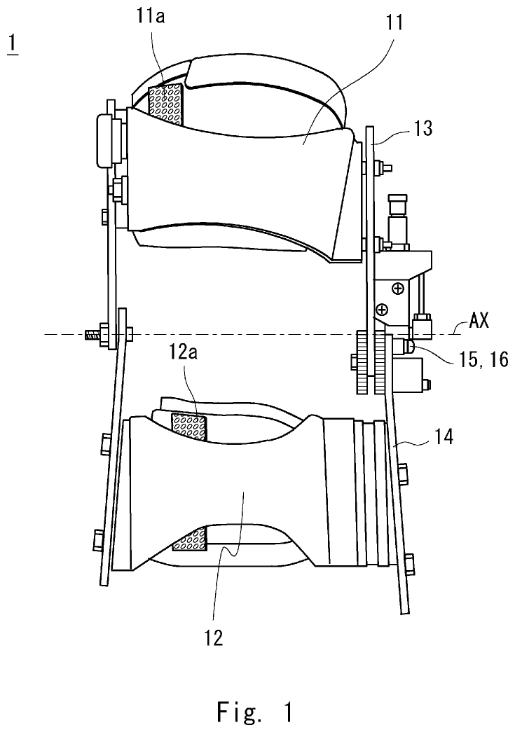

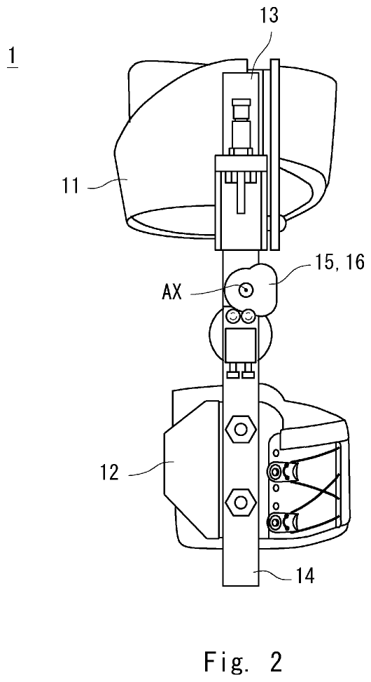

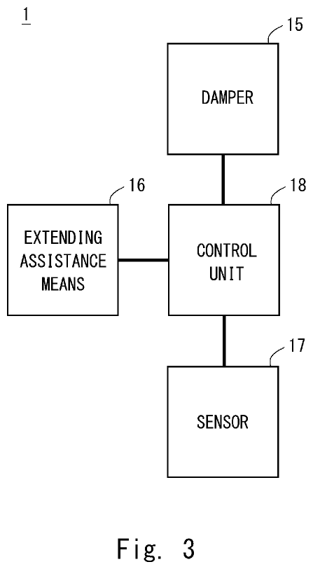

FIG. 1 is a front view illustrating the walking assistance apparatus according to the first embodiment. FIG. 2 is a side view illustrating the walking assistance apparatus according to the first embodiment. FIG. 3 is a block diagram showing a control system of a walking assistance apparatus 1 according to the first embodiment. As shown in FIGS. 1 to 3, the walking assistance apparatus 1 includes an upper leg supporter 11, a lower leg supporter 12, an upper leg frame 13, a lower leg frame 14, a damper 15, extending assistance means 16, a sensor 17, and a control unit 18. A short lower-limb orthosis may be attached to a lower side of the walking assistance apparatus 1. The walking assistance apparatus 1 is attached to the leg of the user. The components of the walking assistance apparatus 1 will be described below.

<Upper Leg Supporter and Lower Leg Supporter>

The upper leg supporter 11 is attached to the upper part of the leg of a user so that it is wound around it, and the lower leg supporter 12 is attached to the lower part of the leg of the user so that it is wound around it. Thus, the upper leg supporter 11 and the lower leg supporter 12 are arranged near the knee joint of the user, specifically, they are arranged over the upper leg and the lower leg of the user. Note that the upper leg indicates a part of the leg from the hip joint to the knee joint, and the lower leg indicates a part of the leg from the knee joint to the ankle joint. The lower leg includes a shin. A part of the leg below the ankle joint, that is, an end part of the leg is a foot.

The upper leg supporter 11 and the lower leg supporter 12 are formed of a stretchable material such as a resin material or a fiber material. The upper leg supporter 11 and the lower leg supporter 12 are wound around the upper leg and the lower leg, respectively, to thereby attach the walking assistance apparatus 1 to the upper leg and the lower leg. The upper leg supporter 11 and the lower leg supporter 12, respectively, may include a hook-and-loop fastener 11a and a hook-and-loop fastener 12a which are attached to the upper leg and the lower leg, respectively. A user winds the upper leg supporter 11 and the lower leg supporter 12 around his/her leg and fixes them with the hook-and-loop fasteners 11a and 12a, respectively.

The hook-and-loop fastener 11a is provided on the front side of the upper leg. The hook-and-loop fastener 12a is provided on the front side of the lower leg. By using the hook-and-loop fasteners 11a and 12a, the user can easily attach and detach the walking assistance apparatus 1. Further, the walking assistance apparatus 1 can be prevented from being displaced from the knee joint of the user. The hook-and-loop fasteners 11a and 12a allow the user to adjust the degree of compression. Further, a fixing band may be provided in order to prevent the hook-and-loop fasteners 11a and 12a from coming off or prevent the upper leg supporter 11 and the lower leg supporter 12 from being displaced.

Note that it is possible to fix the walking assistance apparatus 1 to the leg without using the hook-and-loop fasteners 11a and 12a. For example, the walking assistance apparatus 1 may be fixed to the upper leg and lower leg by using fixing means such as a belt, a button, a pin, or a band. The user can still wear the walking assistance apparatus 1 even when the fixing means described above is employed.

<Upper Leg Frame and Lower Leg Frame>

The upper leg frame 13 is attached to a side part of the upper leg supporter 11. The upper leg frame 13 is disposed along the upper leg. The lower leg frame 14 is attached to a side part of the lower leg supporter 12. The lower leg frame 14 is disposed along the lower leg. The upper leg frame 13 and the lower leg frame 14 are connected to each other through the damper 15 and the extending assistance means 16. Specifically, the damper 15 and the extending assistance means 16 are located at the height of the knee joint so that a rotation axis Ax of the damper 15 and the extending assistance means 16 substantially coincides with the axis of the knee joint. The upper leg frame 13 and the lower leg frame 14 configure a link mechanism that is rotatable about the rotation axis Ax of the damper 15 and the extending assistance means 16.

<Damper>

The damper 15 applies a resisting force in a bending direction of the knee joint of the leg of a user. The bending direction is a direction in which the knee joint is bent in a rotational direction of the knee joint. The damper 15 is, for example, a rotary damper, and is located at a side part of the knee joint. The damper 15 reduces its speed of rotation in the bending direction of the knee joint by using, for example, viscous resistance of a fluid such as oil, elastic resistance of a spring or the like, and frictional resistance of a disk or the like. The damper 15 can switch the resisting force by varying the resisting force. The damper 15 can gradually change the magnitude of the resisting force.

The damper 15 may be a one-way damper that applies a resisting force in only one direction. Therefore, the damper 15 is configured to move freely so as not to apply the resisting force in an extending direction of the knee joint. The extending direction is the direction in which the knee joint extends in the rotational direction of the knee joint. As will be described later, the mode of the damper 15 can be switched by the control unit 18 between a first bending mode and a second bending mode in which the resisting force applied in the bending direction is made greater than that in the first bending mode. The first bending mode may be a free mode in which no resisting force is applied in the bending direction. When the first bending mode is the free mode, the second bending mode is referred to as a damper mode. The second bending mode includes a period in which a magnitude of the resisting force is gradually increased from that of the resisting force in the first bending mode.

<Extending Assistance Means>

The extending assistance means 16 applies an assisting force to the extending direction of the knee joint of the leg. The assisting force is a force that acts on the knee joint in the extending direction so that the upper leg and the lower leg are linearly extended. The extending assistance means 16 is, for example, an actuator and is located on a side of the knee joint. Therefore, the extending assistance means 16 is disposed at a position where the knee can be extended. Like the damper, the extending assistance means 16 is disposed near the axis of the knee joint. Note that the extending assistance means 16 is not limited to the actuator, but may instead be an elastic body such as a spring as long as the force can be applied to the knee joint in the extending direction. The extending assistance means 16 can switch the assisting force by varying the assisting force. The extending assistance means 16 can gradually change the magnitude of the assisting force.

As will be described later, the mode of the extending assistance means 16 can be switched by the control unit 18 between a first extending mode and a second extending mode in which the assisting force applied in the extending direction of the knee joint is made greater than that in the first extending mode. The first extending mode may be a free mode in which no assisting force is applied in the extending direction of the knee joint. When the first extending mode is the free mode, the second extending mode is referred to as an extending assistance mode. The second extending mode includes a period in which the magnitude of the assisting force is gradually increased from that of the assisting force in the first extending mode.

<Control System>

Next, a control system of the walking assistance apparatus 1 is described. As shown in FIG. 3, in the control system of the walking assistance apparatus 1, the sensor 17 and the control unit 18 control the damper 15 and the extending assistance means 16. The damper 15, the extending assistance means 16, and the sensor 17 are connected to the control unit 18 via a wired or wireless communication line. The control unit 18 may be separated from the main body of the walking assistance apparatus 1 including the damper 15 and the extending assistance means 16 and only the communication line may be connected thereto, or the control unit 18 may be attached to the main body together with the damper 15 and the extending assistance means 16.

<Sensor>

The sensor 17 detects a timing in the walking motion of a user. Specifically, the sensor 17 is provided for detecting a switching timing in a gait cycle (a gait frequency). For example, the sensor 17 detects an angle between the ground and the lower leg, and outputs the detected angle to the control unit 18 as a result of the detection. The control unit 18 switches the modes of the damper 15 and the extending assistance means 16 based on the result of the detection by the sensor 17.

Specifically, the control unit 18 switches the modes of the damper 15 and the extending assistance means 16, for example, based on the angle between the ground and the lower leg. That is, the control unit 18 switches the modes of the damper 15 and the extending assistance means 16 based on a timing signal output from the sensor 17. By this configuration, the modes of the damper 15 and the extending assistance means 16 are switched by the control unit 18 at a fixed timing in the gait cycle. Power is supplied to the sensor 17 and the control unit 18 from a battery (not shown) mounted on the walking assistance apparatus 1.

Various types of sensors can be used as the sensor 17. Specific examples of the sensor 17 will be described below.

The sensor 17 may be, for example, a gyro sensor that detects an angle of the lower leg (the shin) with respect to the ground. Further, the sensor 17 may be an angle sensor that detects an angle of the leg, that is, an angle formed by the upper leg and the lower leg around the knee joint. Further, the sensor 17 may be an angle sensor that detects a bending angle at which the lower leg is bent from a state in which the upper leg and the lower leg are linearly extended and the knee joint is thus extended. The sensor 17 may be an angular velocity sensor that detects an angular velocity of an angle of the lower leg, and angular velocities of an angle and a bending angle of the leg.

The sensor 17 may be a distance measuring sensor that detects a distance between a predetermined position of the leg and the ground. The predetermined position of the leg is, for example, a shoe, a foot, a sole of a foot, or the like. For example, the distance measuring sensor attached near a shoe, a foot, or the vicinity thereof can be used as the sensor 17. As the distance from a shoe, a foot, a sole of a foot, or the like to the ground varies in accordance with the walking motion, a waveform corresponding to the gait cycle can be detected. Note that an optical sensor can be used as the distance measuring sensor. Further, the ground includes a floor surface.

The sensor 17 detects a waveform corresponding to the gait cycle from the detected angles, angular velocities, distances, and the like. That is, the detected angles, angular velocities, distances, and the like are periodically changed in accordance with the gait cycle. The sensor 17 detects a walking timing based on the detected angles, angular velocities, distances, and the like.

For example, the control unit 18 may compare the output value of the sensor 17 with a threshold and switch the modes of the damper 15 and the extending assistance means 16 in accordance with the result of the comparison. For example, the control unit 18 switches the modes in accordance with a timing signal indicating the timing at which the output value of the sensor 17 exceeds the threshold or a timing signal indicating the timing at which the output value thereof falls below the threshold.

Note that in the damper 15, a first threshold (e.g., a first angle of the lower leg) for detecting a switching timing at which the modes are switched from the first bending mode to the second bending mode and a second threshold (e.g., a second angle of the lower leg) for detecting a switching timing at which the modes are switched from the second bending mode to the first bending mode may be set. Further, in the extending assistance means 16, a third threshold (e.g., a third angle of the lower leg) for detecting a switching timing at which the modes are switched from the first extending mode to the second extending mode and a fourth threshold (e.g., a fourth angle of the lower leg) for detecting a switching timing at which the modes are switched from the second extending mode to the first extending mode may be set.

Further, the sensor 17 may be a ground contact timing sensor that detects a ground contact timing (i.e., a timing at which the sole of the foot comes into contact with the ground) of the sole of the foot. The sensor 17 can detect the walking timing based on the detected ground contact timing. For example, the sensor 17 detects the switching timing by adding or subtracting in advance the time from the ground contact timing to the switching timing of each mode.

Further, the sensor 17 may be an imaging sensor that shoots the leg. In this case, the sensor 17 shoots the state of the leg from the outside of the walking assistance apparatus 1. The walking timing can be detected (i.e. determined) from the state of the shot leg. Note that when the sensor 17 is disposed outside the walking assistance apparatus 1, the walking assistance apparatus 1 may include a reception unit that receives a signal indicating the switching timing from the external sensor 17.

Further, the switching timing may be detected using a plurality of sensors 17 in combination. For example, the sensor 17 may include both of a first sensor for detecting the angle of the lower leg and a second sensor for detecting the distance from the sole of the foot to the floor surface. As a matter of course, the specific examples of the sensor 17 are not limited to the aforementioned examples. The sensor 17 may be mounted on the walking assistance apparatus 1.

Alternatively, the sensor 17 may be mounted outside the walking assistance apparatus 1.

<Control Unit>

The control unit 18 controls the damper 15 and the extending assistance means 16 based on the switching timing output from the sensor 17. The control unit 18 switches the modes of the damper 15 and the extending assistance means 16.

The control unit 18 switches the modes of the damper 15 so that the first bending mode and the second bending mode are repeated alternately. For example, in the first bending mode, the damper 15 is turned off to be brought into the free mode in which no resisting force is applied in the bending direction. In the second bending mode, the damper 15 is turned on to be brought into the damper mode in which a resisting force is applied in the bending direction. Alternatively, for example, in the first bending mode, the damper 15 is switched so as to apply a small resisting force, and in the second bending mode, the damper 15 is switched so as to apply a large resisting force.

Further, the control unit 18 switches the modes of the extending assistance means 16 so that the first extending mode and the second extending mode are repeated alternately. For example, in the first extending mode, the extending assistance means 16 is turned off to be brought into the free mode in which no assisting force is applied in the extending direction. In the second extending mode, the extending assistance means 16 is turned on to be brought into the extending assistance mode in which an assisting force is applied in the extending direction. Alternatively, for example, in the first extending mode, the extending assistance means 16 is switched so as to apply a small assisting force, and in the second extending mode, the extending assistance means 16 is switched so as to apply a large assisting force.

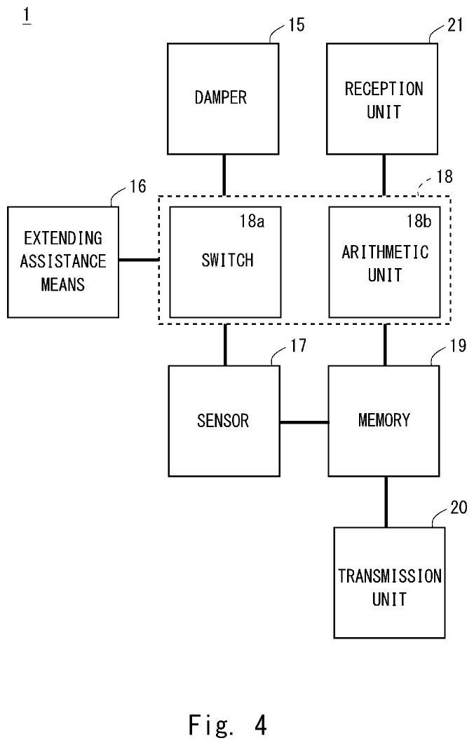

FIG. 4 is a block diagram showing another control system of the walking assistance apparatus 1 according to the first embodiment. In FIG. 4, in addition to the configuration shown in FIG. 3, the walking assistance apparatus 1 includes a memory 19, a transmission unit 20, and a reception unit 21. Further, the control unit 18 includes a switch 18a and an arithmetic unit 18b. Note that the damper 15, the extending assistance means 16, and the sensor 17 are similar to those shown in FIG. 3, and thus the descriptions thereof are omitted.

The arithmetic unit 18b is formed by, for example, hardware mainly using a microcomputer including: a Central Processing Unit (CPU) that performs arithmetic processing, control processing, and the like, a Read Only Memory (ROM) that stores an arithmetic program, a control program, and the like executed by the CPU, a RAM (Random Access Memory) that stores various types of data, and an interface unit (I/F) that performs input/output of signals from/to the outside. The CPU, the ROM, the RAM, and the interface unit are connected to one another through a data bus or the like.

The output value of the sensor 17 is input to the arithmetic unit 18b. The arithmetic unit 18b acquires the switching timing in the gait cycle by performing predetermined arithmetic processing on the output value of the sensor 17. For example, the arithmetic unit 18b acquires the switching timing by comparing the output value of the sensor with a threshold. When the arithmetic unit 18b outputs the acquired switching timing to the switch 18a, the switch 18a switches the modes of the damper 15 and the extending assistance means 16. The memory 19 stores therein the data of the output value of the sensor 17 which is output during the walking training.

A switching signal indicating that the modes have been switched may be input from the switch 18a to the arithmetic unit 18b. The arithmetic unit 18b counts the number of times the switching operations are performed by the switch 18a based on the switching signal. The arithmetic unit 18b counts the number of times the switching operations are performed by the switch 18a and writes this number of switching operations into the memory 19. The number of switching operations performed by the switch 18a corresponds to the number of steps taken by a user. By this configuration, the user can count the number of steps he/she has taken during the walking training without having to wear a pedometer (registered trademark) or the like. Accordingly, the convenience of the walking assistance apparatus can be improved. Further, the user can count the number of steps he/she has taken during the walking training without having to wear an additional sensor for counting the number of steps. Therefore, the cost and the size of the walking assistance apparatus 1 can be reduced in comparison to the configuration in which the sensor for counting the number of steps is additionally provided.

The transmission unit 20 transmits data to an external device, for example, an external server. The reception unit 21 receives data from the external device. The transmission unit 20 and the reception unit 21 transmit/receive data in accordance with a communication standard such as Bluetooth (registered trademark). Both the transmission unit 20 and the reception unit 21 may perform communication by radio communication or wired communication.

The transmission unit 20 transmits data acquired by the walking assistance apparatus 1, such as the output value of the sensor 17 during the walking training and the number of switching operations performed by the switch 18a, to the external device. By doing so, the external device can collect data. This configuration makes it possible for the walking assistance apparatus 1 to cooperate with the external device and to reduce the capacity of the memory 19. Further, the transmission unit 20 may automatically transmit the output of the sensor 17 during the training.

The reception unit 21 receives data from the external device such as the external server. By this configuration, the conditions for detecting the walking timing can be changed. Specifically, the setting of a threshold for detecting the switching timing and the like can be changed. For example, when the setting of the threshold is changed, the external device may transmit an instruction to change the setting of the threshold. When the reception unit 21 receives the instruction to change the setting of the threshold, the arithmetic unit 18b changes the threshold. By this configuration, the most suitable detection conditions can be set for each user. Thus, the most suitable detection conditions can be set for each user.

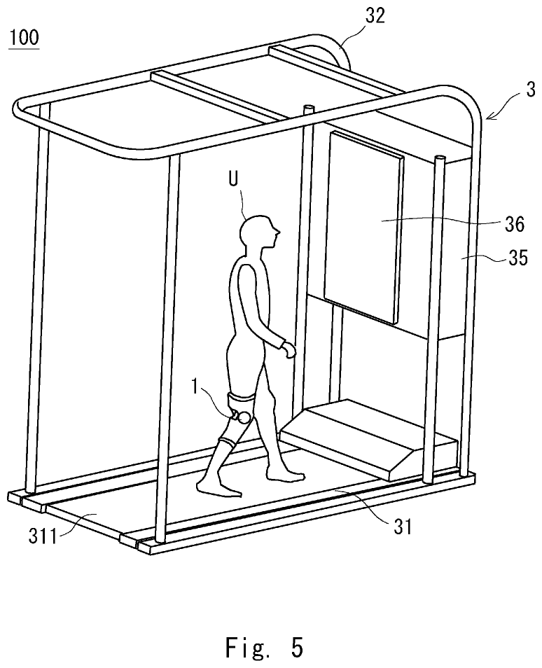

Note that the external device may be a training apparatus 3 of a walking training system 100 as shown in FIG. 5. The walking training system 100 includes the walking assistance apparatus 1 attached to the leg of a user U and the training apparatus 3 that performs walking training of the user U. Further, the training apparatus 3 includes a treadmill 31, a frame main body 32, a control device 35, and a display unit 36. The treadmill 31, the control device 35, and the display unit 36 are fixed to the frame main body 32. The display unit 36 is disposed in front of the user U.

The treadmill 31 includes a rotatable ring-like belt conveyor 311 where the user U walks, and rotates the belt conveyor 311 at a set speed Vs. The user U stands on the belt conveyor 311 and walks thereon in accordance with the movement of the belt conveyor 311. The display unit 36 displays information such as a training instruction to the user U, a training menu, and training information (e.g., the set speed and the biological information). For example, the display unit 36 may include a touch panel, in which case the user U can input various kinds of information through the display unit 36. Further, the training apparatus 3 may include a camera or the like for shooting the user U. By this configuration, the display unit 36 can display an image of the walking motion of the user U during the training.

The control device 35 is formed by, for example, hardware mainly using a microcomputer including: a Central Processing Unit (CPU) that performs arithmetic processing, control processing, and the like, a Read Only Memory (ROM) that stores an arithmetic program, a control program, and the like executed by the CPU, a RAM (Random Access Memory) that stores various types of data, and an interface unit (I/F) that performs input/output of signals from/to the outside. The CPU, the ROM, the RAM, and the interface unit are connected to one another through a data bus or the like.

The control unit 18 of the walking assistance apparatus 1 may transmit and receive various types of data to and from the training apparatus 3 through the transmission unit 20 and the reception unit 21. Further, the control device 35 of the training apparatus 3 may have the function of the control unit 18 of the walking assistance apparatus 1.

<Operation>

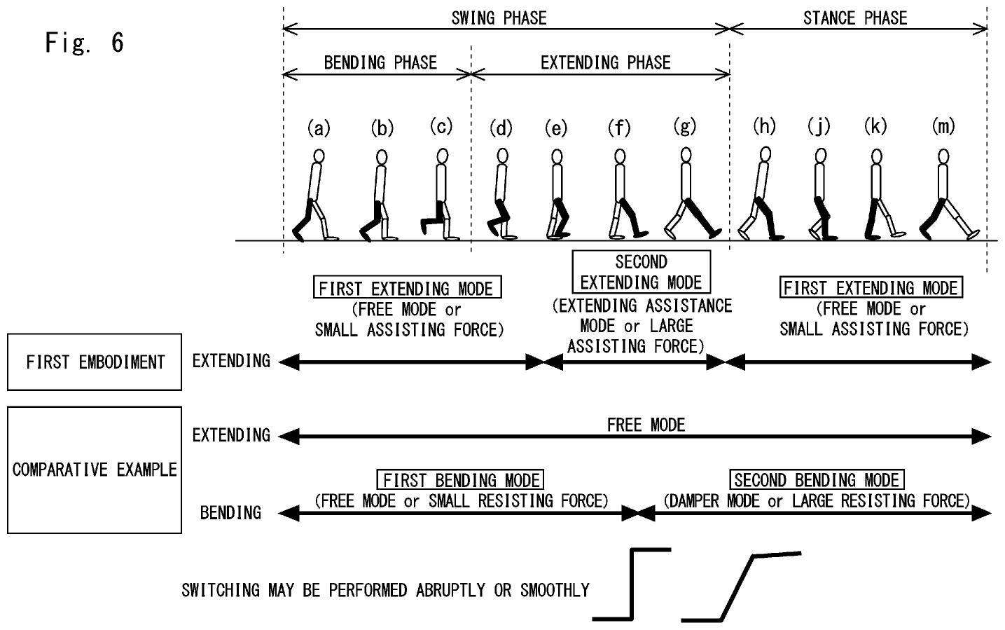

Next, an operation of the walking assistance apparatus 1 is described. FIG. 6 is a diagram illustrating a walking motion and a timing at which the modes are switched in the gait cycle using the walking assistance apparatus 1 according to the first embodiment. FIG. 6 also shows a comparative example. First, one gait cycle including a swing phase and a stance phase is described with reference to FIG. 6. Next, a walking motion and a timing at which the modes are switched in the comparative example will be described. After that, the walking motion and the timing at which modes are switched in this embodiment in comparison with those in the comparative example will be described.

<One Gait Cycle>

As shown in FIG. 6, one gait cycle includes two steps, that is, one left-leg step and one right-leg step. In FIG. 6, one gait cycle is shown in the order of timings (a) to (m). After the timing (m), the timing returns to the timing (a) to start the next gait cycle. In FIG. 6, the timings from (a) to (g) are in a swing phase, and the timings from (h) to (m) are in a stance phase.

In the swing phase, the sole of the foot of the leg to which the walking assistance apparatus 1 is attached comes off the ground, and in the stance phase, the sole of the foot of the leg to which the walking assistance apparatus 1 is attached is in contact with the ground. Between the timing (g) and the timing (h), the sole of the foot comes into contact with the ground, and between the timing (m) back to the timing (a), the sole of the foot comes off the ground. The timings (a) to (c) are in a bending phase during which a bending angle of the knee joint is increased, and the timings (d) to (g) are in an extending phase during which the bending angle of the knee joint is reduced. Note that the swing phase, the stance phase, the bending phase, and the extending phase are set based on the diseased leg to which the walking assistance apparatus 1 is attached.

### Comparative Example: Bending Direction

Next, a walking motion and a timing at which the modes are switched in the one gate cycle in the comparative example is described. First, the operation of the damper 15 that applies a resisting force in the bending direction of the knee joint is described. In the comparative example, the mode of the damper 15 is switched from the first bending mode to the second bending mode in the extending phase, specifically, at the timing (f). Further, the mode of the damper 15 is switched from the second bending mode to the first bending mode at the timing when the phase is changed from the stance phase to the swing phase, specifically, between the timing (m) and the timing (a).

The first bending mode includes a free mode in which a resisting force is not applied in the bending direction of the knee joint or a mode in which a resisting force smaller than that in the second bending mode is applied in the bending direction of the knee joint. The second bending mode includes a damper mode in which a resisting force is applied in the bending direction of the knee joint or a mode in which a resisting force larger than that in the first bending mode is applied in the bending direction of the knee joint.

In the swing phase, it is not necessary to support the weight of a user with the diseased leg. Therefore, in the swing phase, it is not necessary to generate a resisting force with respect to the knee joint by the damper 15, or it is sufficient to generate a small amount of resisting force even when it is necessary to generate the resisting force. Thus, during most of the swing phase, in regard to the bending direction, the control unit 18 can set the mode to the first bending mode including the free mode. Meanwhile, during the entire stance phase, the control unit 18 sets the mode to the second bending mode, where a resisting force is generated with respect to the damper 15 in the bending direction.

Further, in the one gate cycle, only the first and the second bending modes are set, and the control unit 18 alternately switches the first and the second bending modes during the walking motion. That is, the control unit 18 controls the damper 15 based on the timing signal. For example, the control unit 18 performs on-off control of the free mode and the damper mode. By doing so, it is possible to perform an appropriate control with a simple configuration. For example, when the walking motion varies between each gait cycle, the switching timings detected by the sensor 17 may vary. Even in this case, at the timing before the phase is changed from the swing phase to the stance phase, that is, at any timing in the extending phase, it is only necessary to switch the mode of the damper 15 from the first bending mode to the second bending mode.

Specifically, it is only necessary for the control unit 18 to switch the modes at a predetermined timing between the timings (d) to (g). Since the margin for the errors in the switching timings detected by the sensor 17 can be widened, an appropriate control can be performed. Further, in the extending phase, the knee joint is gradually rotated in the extending direction from a bending state. Therefore, even when the damper 15 is in the second bending mode, no resisting force is generated. Thus, it is possible to switch the modes without the walking motion of a user being interrupted by the dumper 15.

Further, the damper 15 moves freely and thus generates no resisting force in the extending direction of the knee joint. Therefore, a user can freely extend the knee joint. Further, as there is no lock mode for locking the damper 15 between the second bending mode including the damper mode and the first bending mode including the free mode, it is possible to prevent the damper 15 from interrupting the walking motion. As the mode is changed from the second bending mode to the first bending mode without being changed to the lock mode, it is possible to easily perform an appropriate control.

The switching timings between the swing phase and the stance phase in accordance with the outputs of the sensor 17 may be detected, so that the first bending mode is set for the swing phase and the second bending mode is set for the stance phase. That is, the timing at which the phase is changed from swing phase to the stance phase and the timing at which the phase is changed from the stance phase to the swing phase may be set to the switching timings of the modes of the damper 15.

Further, the switching between the first and the second bending modes may be performed abruptly or smoothly. That is, the second bending mode may include a period in which a magnitude of a resisting force is gradually increased from that of the resisting force in the first bending mode, or a period in which a magnitude of a resisting force is gradually reduced until it becomes that of the resisting force in the first bending mode.

### Comparative Example: Extending Direction

Next, a motion of the knee joint in the extending direction in the comparative example is described. In the comparative example, the extending assistance means 16 is not provided or the extending assistance means 16 is not operated. Further, the damper 15 generates no resisting force in the extension direction of the knee joint. Thus, in the comparative example, the knee joint can be moved freely in the extension direction during the entire gait cycle.

In the comparative example, when a person who walks while bending his/her knees, a person whose knees are already bent in the initial stage of the stance phase, or the like uses the walking assistance apparatus, a strong moment torque may be applied to his/her lower limbs from the initial stage of the stance phase. Such a case causes a problem that the capacity of the damper 15 for generating a resisting force to a rotation of the knee joint in the bending direction is exceeded, and thus the knee is easily bent.

### Embodiment: Bending Direction

Next, an operation of the walking assistance apparatus 1 according to this embodiment is described. In this embodiment, the operation of the damper 15 that applies a resisting force in the bending direction of the knee joint is similar to that of the comparative example, and thus the descriptions thereof are omitted.

### Embodiment: Extending Direction

Next, an operation of the extending assistance means 16 that applies an assisting force in the extending direction of the knee joint in this embodiment is described. In this embodiment, unlike the comparative example, the operation of the extending assistance means 16 in the extending direction of the knee joint includes the first extending mode and the second extending mode. The first extending mode and the second extending mode are alternately switched. Specifically, in the extending phase, for example, at the timing (e), the mode of the extending assistance means 16 is switched from the first extending mode to the second extending mode.

In this way, in the swing phase during which the sole of the foot of the leg comes off the ground, the control unit 18 switches the mode of the extending assistance means 16 from the first extending mode to the second extending mode at least in a later stage of the extending phase during which the knee joint comes close to an extended state. Specifically, the control unit 18 switches the mode of the extending assistance means 16 from the first extending mode to the second extending mode in a later stage of the extending phase during which the bending angle of the lower leg is reduced. Note that the control unit 18 can adjust a start timing of the second extending mode in accordance with the degree of a bending of the knee joint of a user (i.e., how much the knee joint of a user can be bent).

For example, for a user whose knee joint can hardly be bent, the control unit 18 starts the second extending mode at an early timing (e.g., at the timing (e)). On the other hand, for a user whose knee joint can be almost normally bent, the control unit 18 starts the second extending mode at the timing just before the stance phase (e.g., at the timing of (g)). For a user whose knee joint can be bent to some degree, the control unit 18 starts the second extending mode, for example, at the timing (f). The control unit 18 can adjust the start timing of the second extending mode by setting it in advance. For example, the start timing can be adjusted by setting the start timing derived from the degree of the bending of the knee joint in advance.

Further, the control unit 18 can adjust the start timing of the second extending mode based on a walking state detected by the sensor 17. The control unit 18 may adjust the start timing while a user is walking based on the various angles, the angular velocities, the distances, and the like detected by the sensor 17.

Meanwhile, the control unit 18 switches the mode of the extending assistance means 16 from the second extending mode to the first extending mode between the timings at which the phase is changed from the swing phase to the stance phase, specifically, between the timing (g) and the timing (h).

Further, the switching between the first and the second extending modes may be performed abruptly or smoothly. That is, the second extending mode may include a period in which a magnitude of an assisting force is gradually increased from that of the assisting force in the first extending mode, or a period in which a magnitude of an assisting force is gradually reduced until it becomes that of the assisting force in the first extending mode.

Next, effects of this embodiment are described. In the walking assistance apparatus 1 according to this embodiment, the extending assistance mode is set before the stance phase and in a later stage of the extending phase. Therefore, an extending assistance is performed in the extending phase with regard to, for example, a person who walks while bending his/her knees or a person whose knees are already bent in an initial stage of the stance phase, and thus it is possible to prevent an excessive knee bending.

Further, the walking assistance apparatus 1 according to this embodiment performs an extending assistance during the swing phase, that is, during a period in which it is not necessary to support the weight of a user with the leg to which the walking assistance apparatus 1 is attached. Further, it is sufficient to perform the extending assistance so that the lower leg is swung once. Therefore, the extending assistance means 16 may be a compact actuator or the like. This configuration makes it possible to reduce the size and the weight of the walking assisting apparatus 1.

It may be considered to use a larger damper to prevent an excessive knee bending when a walking assistance is performed with regard to, for example, a person who walks while bending his/her knees or a person whose knees are already bent in an initial stage of the stance phase. However, when a walking assistance apparatus includes such a large damper mounted thereon, the weight of this walking assistance apparatus is increased and consequently it becomes a large walking assistance apparatus. It is difficult for a hemiplegic patient to support such a large walking assistance apparatus with his/her paralyzed leg. Further, it is necessary to take measures to prevent the walking assistance apparatus from slipping off the leg where it is attached. On the other hand, in this embodiment, the extending assistance means 16 using a compact actuator or the like can be used, so that the size and the weight of the walking assistance apparatus 1 can be reduced.

Note that the control of the mode switching between the first and the second bending modes and the control of the mode switching between the first and the second extending modes are not limited to the on-off controls. The magnitude of the resisting force of the damper 15 and the magnitude of the assisting force of the extending assistance means 16 may be made to gradually increase or decrease at the timings at which the modes are switched. By this configuration, it is possible to make the walking motion of a user smooth.

Further, the walking assistance apparatus 1 may include a battery for supplying power mounted thereon. The battery may be a secondary battery that can be charged/discharged, or may be a primary battery. Further, the battery may be a solar cell. In this case, it is only necessary to provide a light receiving part for the solar cell on a surface of the walking assistance apparatus 1. Further, the control unit 18 may include the switch 18a such as a solenoid switch or a semiconductor switch. The control unit 18 may include an electronic circuit, and switch the modes of the damper 15 and the extending assistance means 16 by controlling the ON/OFF switching of the switch 18a in accordance with a control signal from the electronic circuit.

### Second Embodiment

Next, a walking assistance apparatus according to a second embodiment is described. In this embodiment, a third bending mode in which the first bending mode or the second bending mode can be selected, and a third extending mode in which the first extending mode or the second extending mode can be selected are provided. The configuration of the walking assistance apparatus according to this embodiment is similar to that of the first embodiment, and thus the descriptions thereof are omitted.

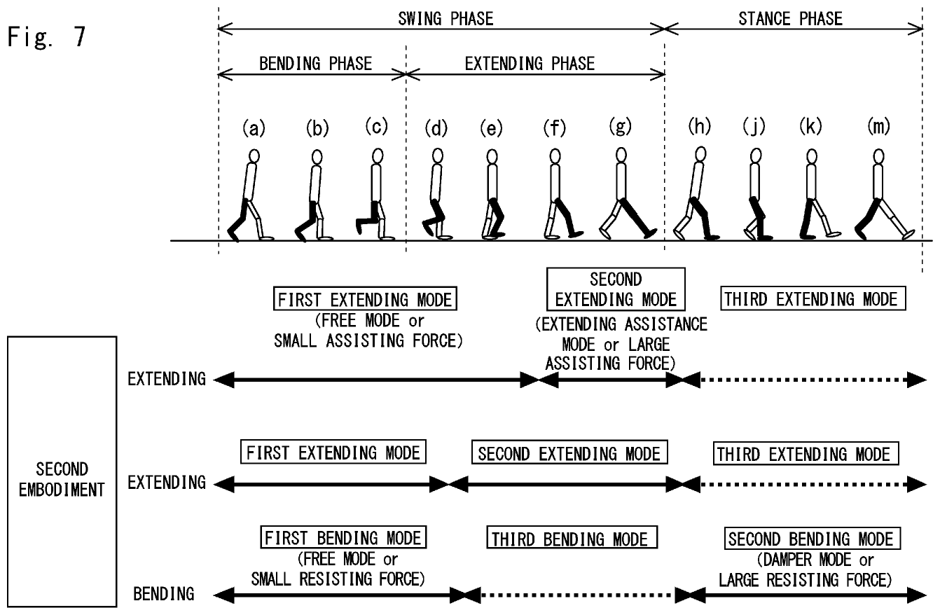

FIG. 7 is a diagram illustrating a walking motion and a timing at which the modes are switched in a gait cycle using the walking assistance apparatus according to the second embodiment. As shown in FIG. 7, in the second embodiment, in regard to the bending direction, the mode is switched from first bending mode to the third bending mode in the extending phase. Specifically, the mode is switched from first bending mode to the third bending mode between the timing (d) and the timing (e). Further, the mode is switched from the third bending mode to the second bending mode at the timing at which the phase is changed from the swing phase to the stance phase, specifically, between the timing (g) and the timing (h).

The third bending mode is a mode in which the first bending mode or the second bending mode can be selected. For example, the third bending mode may be a free mode or a damper mode. In the extending phase, the first bending mode or the second bending mode is set in the damper 15. Further, the mode is switched from the third bending mode to the second bending mode at the timing at which the phase is changed from the stance phase to the swing phase, specifically, between the timing (m) and the timing (a).

In the extending direction, the mode is switched from the second extending mode to the third extending mode in the stance phase. Specifically, the mode is switched from the second extending mode to the third extending mode between the timing (g) and the timing (h). Therefore, in the stance phase during which the sole of the foot is in contact with the ground, the first bending mode or the second bending mode is set in the extending assistance means 16.

Note that the timing at which the mode is switched from the first extending mode to the second extending mode may be the timing (e) or the timing (f) as in the case of the first embodiment. Alternatively, it may be the timing (g).

According to this embodiment, the extending assistance means 16 of the walking assistance apparatus of this embodiment has the third extending mode which may be the free mode or the extending assistance mode in the stance phase. The extending assistance means 16 does not require torque to support the body weight in the first place. Thus, during the stance phase, the extending assistance means 16 does not affect the walking even if it is operated. Therefore, the control unit 18 can change the aforementioned modes depending on the situation.

Note that the present disclosure is not limited to the above-described embodiments and can be modified as appropriate without departing from the spirit of the present disclosure. For example, a walking assistance method and a walking assistance program using the walking assistance apparatus attached to the leg of a user are within the scope of the technical ideas of the above-described embodiments.

The program can be stored and provided to a computer using any type of non-transitory computer readable media. Non-transitory computer readable media include any type of tangible storage media. Examples of non-transitory computer readable media include magnetic storage media (such as floppy disks, magnetic tapes, hard disk drives, etc.), optical magnetic storage media (e.g., magneto-optical disks), CD-ROM (compact disc read only memory), CD-R (compact disc recordable), CD-R/W (compact disc rewritable), and semiconductor memories (such as mask ROM, PROM (programmable ROM), EPROM (erasable PROM), flash ROM, RAM (random access memory), etc.). The program may be provided to a computer using any type of transitory computer readable media. Examples of transitory computer readable media include electric signals, optical signals, and electromagnetic waves. Transitory computer readable media can provide the program to a computer via a wired communication line (e.g., electric wires, and optical fibers) or a wireless communication line.

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

What is claimed is:

1. A walking assistance system attached to a leg of a user, comprising:
a damper configured to apply a resisting force in a bending direction of a knee joint of the leg;
extending assistance means for applying an assisting force in an extending direction of the knee joint;
a sensor configured to detect a switching timing in a gait cycle of the user; and
a control unit configured to switch modes of the damper in accordance with the switching timing so that a first bending mode and a second bending mode in which a resisting force larger than that in the first bending mode is applied are alternately repeated and switch modes of the extending assistance means in accordance with the switching timing so that a first extending mode and a second extending mode in which an assisting force larger than that in the first extending mode is applied are alternately repeated,
wherein in a swing phase during which a sole of a foot of the leg comes off a ground, the control unit switches the mode of the extending assistance means from the first extending mode to the second extending mode at least in a later stage of an extending phase during which the knee joint comes close to an extended state.

2. The walking assistance system according to claim 1, wherein the control unit is configured to be able to adjust a start timing of the second extending mode in accordance with a degree of a bending of the knee joint of the user.

3. The walking assistance system according to claim 1, wherein the control unit is configured to be able to adjust the start timing of the second extending mode by setting it in advance.

4. The walking assistance system according to claim 1, wherein the control unit is configured to be able to adjust the start timing of the second extending mode based on a walking state detected by the sensor.

5. The walking assistance system according to claim 1, wherein the sensor comprises any one of: a gyro sensor configured to detect an angle of a lower leg with respect to the ground; an angle sensor configured to detect an angle formed by an upper leg and the lower leg around the knee joint; an angle sensor configured to detect a bending angle at which the lower leg is bent from a state in which the knee joint is extended; an angular velocity sensor configured to detect an angular velocity of each of the angles or an angular velocity of the bending angle; a distance measuring sensor configured to detect a distance between a predetermined position of the leg and the ground; a ground contact timing sensor configured to detect a timing at which the sole of the foot comes into contact with the ground; and an imaging sensor configured to shoot the leg.

6. The walking assistance system according to claim 1, wherein the second bending mode includes a period in which a magnitude of the resisting force is gradually increased from that of the resisting force in the first bending mode.

7. The walking assistance system according to claim 1, wherein the second extending mode includes a period in which a magnitude of the assisting force is gradually increased from that of the assisting force in the first extending mode.

8. The walking assistance system according to claim 1, wherein the extending assistance means comprises an actuator or an elastic body.

9. The walking assistance system according to claim 1, wherein in a stance phase during which the sole of the foot is in contact with the ground, the first extending mode or the second extending mode is set in the extending assistance means.

10. The walking assistance system according to claim 1, wherein in the extending phase, the first bending mode or the second bending mode is set in the damper.

11. A walking assistance method using a walking assistance apparatus attached to a leg of a user,
the walking assistance apparatus comprising:
a damper configured to apply a resisting force in a bending direction of a knee joint of the leg;
extending assistance means for applying an assisting force in an extending direction of the knee joint; and
a sensor configured to detect a switching timing in a gait cycle of the user,
the walking assistance method comprising switching modes of the damper in accordance with the switching timing so that a first bending mode and a second bending mode in which a resisting force larger than that in the first bending mode is applied are alternately repeated and switching modes of the extending assistance means in accordance with the switching timing so that a first extending mode and a second extending mode in which an assisting force larger than that in the first extending mode is applied are alternately repeated,
wherein in a swing phase during which a sole of a foot of the leg comes off a ground, the mode of the extending assistance means is switched from the first extending mode to the second extending mode at least in a later stage of an extending phase during which the knee joint comes close to an extended state.

12. A non-transitory computer readable medium storing a walking assistance program using a walking assistance apparatus attached to a leg of a user,
the walking assistance apparatus comprising:
a damper configured to apply a resisting force in a bending direction of a knee joint of the leg;
extending assistance means for applying an assisting force in an extending direction of the knee joint; and
a sensor configured to detect a switching timing in a gait cycle of the user,
the walking assistance program causing a computer to switch modes of the damper in accordance with the switching timing so that a first bending mode and a second bending mode in which a resisting force larger than that in the first bending mode is applied are alternately repeated and switch modes of the extending assistance means in accordance with the switching timing so that a first extending mode and a second extending mode in which an assisting force larger than that in the first extending mode is applied are alternately repeated,
wherein in a swing phase during which a sole of a foot of the leg comes off a ground, the mode of the extending assistance means is switched from the first extending mode to the second extending mode at least in a later stage of an extending phase during which the knee joint comes close to an extended state.
